(19) United States
(12) Patent Application Publication
(10) Pub. No.: US 2004/0197836 A1
(43) Pub. Date: Oct. 7, 2004

(54) Measurement of F-actin in whole blood cellular subsets

(76) Inventor: Brian B. Hashemi, Houston, TX (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/407,262

(22) Filed: Apr. 4, 2003

(51) Int. Cl. G01N 33/567; C12P 19/00

(52) U.S. Cl. 435/7.21

(57) ABSTRACT

A method for F-actin measurement in a mixture of cells includes fixing the mixture of cells at a selected temperature; labeling at least one type of cells in the mixture of cells using cell type-specific reagents; labeling F-actin using an F-actin probe; and determining a content of the F-actin in each of the at least one type of cell in the mixture of cells. The method may further include incubating separate samples with or without a stimulant before the fixing of the mixture of cells; and comparing the F-actin contents in the same cell types in the separate samples.

### STATEMENT REGARDING FEDERALLY SPONSORED RESEARCH OR DEVELOPMENT

This application results from a study supported by NASA Grant NAG-21357. The government has certain rights in the invention.

### BACKGROUND OF INVENTION

1. Field of the Invention

The invention relates to methods for measuring F-actin contents in cells, particularly, measurements of F-actin contents in a mixture of cells.

2. Background Art

Actin is a globular protein (G-actin) with an Adenosine Triphosphate (ATP) binding site in the center of the molecule. The monomeric G-actin may dimerize or form trimers; these dimers or trimers may serve as the nucleation sites for the formation of filamentary Actin (F-actin). Actin polymerization requires the presence of ATP, magnesium (Mg) and potassium (K). The concentration of G-actin is also important. Above a critical concentration of G-actin, the G-actin molecules will polymerize, while F-actin will de-polymerize when G-actin concentration is below the critical concentration.

Actin is an abundant protein, and polymerized F-actin filaments form the cytoskeleton structures inside the cells. The cytoskeleton both affects and is affected by various functions and processes of the cells. For example, cytoskeletal structures and their resulting motile and mechanical functions play important roles in cellular function including mitogenesis, cytokinesis, secretion of signaling molecules, cell growth, and initiation of DNA synthesis (Ingber, 1997, Annual Review of Physiology 59: 575-599; Cantrell, 2000, Ann. Rev. Immunol. 18: 165-84; Brock and Chrest, 1993, J. Cell Physiol. 157(2): 367-78; Bunnell et. al., 2001, Immunity 14(3): 315-29; Dustin and Cooper, 2000, Nat. Immunol. 1(1): 23-9; Entschladen and Zanker, 2000, J. Cancer Res. Clin. Oncol. 126(12): 671-81; Gomez et al., 1995, Eur. J. Immunol. 25(9): 2673-8

In addition, the actin cytoskeleton plays a crucial role in orchestrating force balance inside the cells, and mammalian cells rely on actin polymerization and de-polymerization for many cellular processes, from signal transduction to functional responses. For example, polymerization and de-polymerization of actins not only defines a T-cell's morphological development and movement, but also plays an important role in facilitating signal transduction during T cell activation (Destin et. al., 2000, Nature Immunol., 1(1): 23).

While the present invention is applicable to a wide variety of cells from a variety of species (including human and animals), the following description will use T lymphocytes and/or other blood cells of humans as examples to illustrate the methods of the invention. T-lymphocytes play a central role in modulating various aspects of the human immune function (S. G. J. V. Nossal, 1993, Sci. Am., 269(3): 53-62; H. M. Grey, 1989, Sci. Am., 251(5): 56-64). They can be activated to produce lymphokines, to proliferate, and, in some instances, to kill target cells. Because of their critical roles, T-cell growth and differentiation are rigorously regulated.

Actin cytoskeletons play a critical role in the normal T cell functions. For example, T-cell antigen receptor complex (TCR), which plays a central role in the recognition of extracellular signals and in the subsequent transmembrane signaling (Weiss and Littman, 1994, Cell, 76(2): 263-74; Schwartz, 1992, Cell, 71(7): 1065-68; Janeway, 1992, Ann. Rev. Immunol., 10: 645-74), depends on actin cytoskeleton for its function. TCR engagement and aggregation on the plasma membrane leads to the activation of cytoskeletal systems that orchestrate the T cell responses, including intracellular signal transduction. The activation of cytoskeletal systems by TCR engagement and aggregation on the surface of T cells induces a rapid increase in cellular F-actin contents (Phatak and Packman, 1994, J. Cell. Physiol., 159: 365-70), as well as reorganization and translocation of spectrin to the plasma membrane with a patch-like distribution (Black et al., 1988, J. Cell Biol., 106: 97-109; Lee et al., 1988, Cell, 55: 807-16). Furthermore, activation of T cells by antigen-presenting cells (APC) is accompanied by the accumulation of actin at the contact site (Pardi et al,. 1992, J. Cell. Biol., 116(5): 1211-20; Ryser, 1982, J. Immunol., 128: 1159-62). Indeed, a fully-functional actin cytoskeleton is required for the prolonged occupancy of T-cell receptor by the APC and the sustained intracellular signaling (Valitutti et al., 1995, J. Exp. Med., 181: 577-84), an event that is a prerequisite to the activation of T cell responses (Hashemi et al, 1996, J. Immunol., 156: 3660-67; Testi et al., 1989, J. Immunol., 142(6): 1854-60; Poenie et al., 1987, EMBO J., 6(8): 2223-32).

Circulating T-cells are normally maintained in a resting (or G₀) state of the cell cycle. Activation by the ligand-TCR interactions causes these G₀ cells to re-enter the cell cycle, which sends the activated cells on their way to progress along a programmed cellular activation cascade, and ultimately to cell proliferation. Actin cytoskeleton is also involved in these processes.

In addition to T cells, actin cytoskeleton is also important for the function of other cells. For example, it is well known that neutrophils respond to chemotatic agents, such as FMLP (N-formyl-methionine-lecine-phenylalanine), and such responses are accompanied by dramatic changes in the cytoskeleton including an increase in F-Actin. (Chen et al., 1996, Intl. Arch. Allergy Immunol., 110: 325-331; Tabor et al., 1998, Kidney Intl., 53: 783-89). Similarly, monocytes respond to lipopolysaccharide (LPS) binding with an increase in cellular F-Actin.

Because actin cytoskeleton is involved in various aspects of cellular functions, the states of the actin cytoskeleton (e.g., the F-actin contents) in the cells can be used as “molecular signatures” of the cells to assess their status and responsiveness to the stimulants. Therefore, it is desirable to have convenient methods for measuring the polymerization states of actin (e.g., F-actin contents) in selected cells in a mixture of cells, e.g., T-lymphocytes, neutrophils, and monocytes in whole blood.

### SUMMARY OF INVENTION

In one aspect, embodiments of the invention relate to methods of measuring cellular F-actin contents in a mixture of cells. A method according to one embodiment of the invention includes fixing the mixture of cells at a selected temperature in a range of 30 to 40 degrees Celsius; labeling at least one type of cell in the mixture of cells using cell type-specific reagents; labeling F-actin using an F-actin probe; and determining a content of the F-actin in each of the at least one type of cell in the mixture of cells.

Another aspect of the invention relates to methods for using F-actin measurement to study cellular responses to cellular stimulants. A method of measuring an effect of a cellular stimulant in a mixture of cells includes incubating an experimental sample of the mixture of cells with the cellular stimulant in a selected buffer at a selected temperature in a range of 30 to 40 degrees Celsius; incubating a control sample of the mixture of cells in the selected buffer without the cellular stimulant at the selected temperature; fixing the experimental sample and the control sample separately at the selected temperature; labeling at least one type of cells in the experimental sample and the control sample separately using cell type-specific reagents; labeling F-actin in the experimental sample and the control sample separately using an F-actin probe; determining a content of the F-actin in each of the at least one type of cell in the experimental sample and the control sample separately; comparing the content of the F-actin in the each of the at least one type of cell in the experimental sample with the content of the F-actin in a corresponding cell in the control sample.

Other aspects of the invention will become apparent from the following description, the drawings, and the claims.

### BRIEF DESCRIPTION OF DRAWINGS

FIG. 1. shows the effect of temperature on cellular F-actin contents.

FIG. 2 shows the effect of calcium ion concentrations on F-actin polymerization in T-cells.

FIG. 3A shows the difference in background F-actin contents in Jurkat T cells as a function of gravitation force.

FIG. 3B shows that activation-induced polymerization of actin is dramatically reduced in Jurkat T cells in microgravity culture.

FIG. 4 shows a flow chart of F-actin measurement method according to one embodiment of the invention.

FIG. 5 shows a flow chart of F-actin measurement used to study cellular responses to stimuli according to another method of the invention.

FIG. 6 shows F-actin contents in subsets of blood cells in a whole blood sample.

FIGS. 7A-7C show time course studies of various cell types activated by OKT3, which is a monoclonal antibody to the T3 antigen of human T cells.

FIG. 8 shows that data form multiple donors may be plotted to analyze donor-donor variability of F-actin and its response to activation.

### DETAILED DESCRIPTION

The present invention relates to methods for measuring F-actin contents and for assessment of cellular responses to stimuli. Embodiments of the invention involve minimal manipulation of cells and do not require the purification of cellular subsets. The methods described herein minimize F-actin measurement artifacts (e.g., due to temperature changes or calcium ion concentration shifts) by rapid stabilization of cells through fixation at a temperature at or near the physiological temperature (37° C.) and at a calcium concentration near that of a normal physiological state. Particular embodiments of the invention are capable of determining the polymerization state of the actin cytoskeletons for each cellular subset in a mixture of cells, permitting measurement of F-actin contents as “molecular signatures” of the cells. With this capability, particular embodiments of the invention permit rapid evaluation of the status of a mixture of cells (such as blood cells in whole blood) and their responsiveness to stimulating agents. These embodiments use flow cytometry or other suitable techniques to gate on (select) the cellular subsets of interest and to measure the cellular F-actin contents within these cells without having to isolate these cells from the mixture. Methods of the invention can be used to characterize the F-actin cytoskeleton in any combination of cells from a variety of species, e.g., various components of human or animal blood. However, to illustrate the utility of the invention, the following description will illustrate, as an example, the application of one method in accordance with the present invention of assessing F-actin contents in various blood cells (e.g., T cells, neutrophils, and monocytes) of the human immune system. One of ordinary skill in the art would appreciate that embodiments of the invention may be applied to any mixture of cells, including non-blood cells, from human or other animals.

Prior art studies of F-actin are typically performed with purified cells that have been extensively processed. The purification process may introduce artifacts. For example, it is known that actins are highly sensitive to temperature and calcium ion concentration changes.

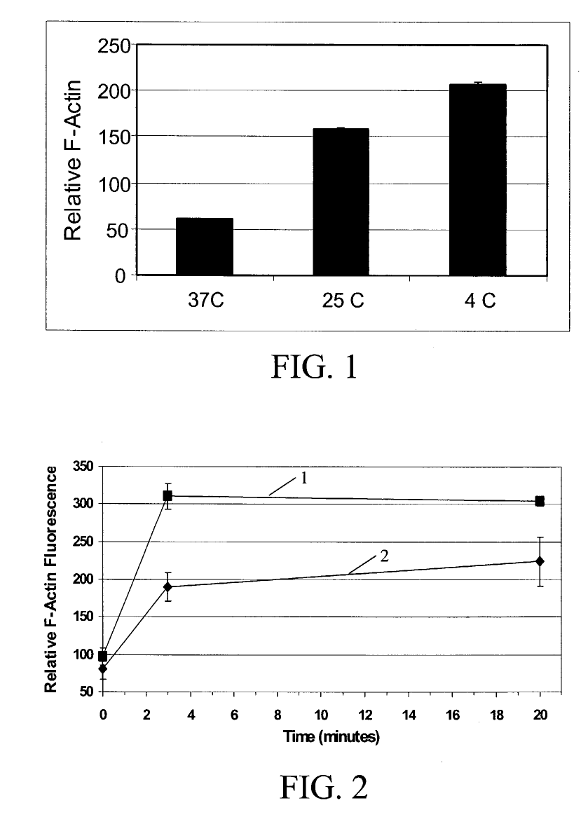

FIG. 1 shows the effect of temperature on cellular F-actin contents. In this example, purified T cells were incubated at the indicated temperatures (4° C., room temperature (25° C.), and 37° C., respectively) for approximately 30 minutes. Cells were then fixed at the indicated temperatures and labeled with a fluorescent F-actin probe. The relative F-actin contents were measured by flow cytometry. These data demonstrate that the polymerization states of actin are highly sensitive to temperature, and at a temperature lower than 37° C., there is a significant increase in the cellular F-actin contents. Therefore, to accurately determine the F-actin contents, it is desirable that cells are stabilized by fixation at 37° C. to preserve the physiological state of F-actin.

FIG. 2 shows the effect of calcium ion concentrations on the inducibility of actin polymerization in T cells. In this example, whole blood was collected from a donor in collection tubes containing heparin (curve 1) or EDTA (curve 2), respectively, as the anticoagulants. Blood samples were then incubated at 37° C. and activated with phorbol ester and a calcium ionophore (PDBu/I, phorbol-12,13-dibutyrate/ionomycin). FIG. 2 shows that chelation of calcium by EDTA results in a dramatic decrease in the responsiveness of T-cells as evidenced by the lower level of inducible actin polymerization by the calcium ionophore. Therefore, to reliably measure the F-actin contents of cells, blood samples should be collected with non-chelating anticoagulants, e.g., heparins or heparinoids.

In addition to the artifacts arising from non-physiological temperatures and altered calcium concentrations, other factors in purification processes may also affect the accuracy of the F-actin measurements, e.g., centrifugal forces. Prior art immune studies are typically based on cellular immunology studies where immune cells are isolated and characterized in tissue cultures. A typical experiment in these studies begins with isolation of a blood sample from a patient or a test subject, followed by steps to separate specific cell types. A typical procedure for separation of cells, for example, may use a nonionic synthetic polymer gradient (such as that sold by Pharmacia Fine Chemicals, Inc. (Piscataway, N.J.) under the trade name of Ficoll™) to isolate “Buffy Coats,” which are enriched in T-cells and monocytes but are mostly devoid of red blood cells. This technique requires layering of the blood sample on a dense medium (e.g., Ficoll™) followed by centrifugation (typically at 300 g) to separate blood components according to their differing densities. Following the purification steps, the isolated cells are then placed in tissue culture and used to assess their responses to stimulating agents (e.g., lectin activation for proliferative responses in T-cells).

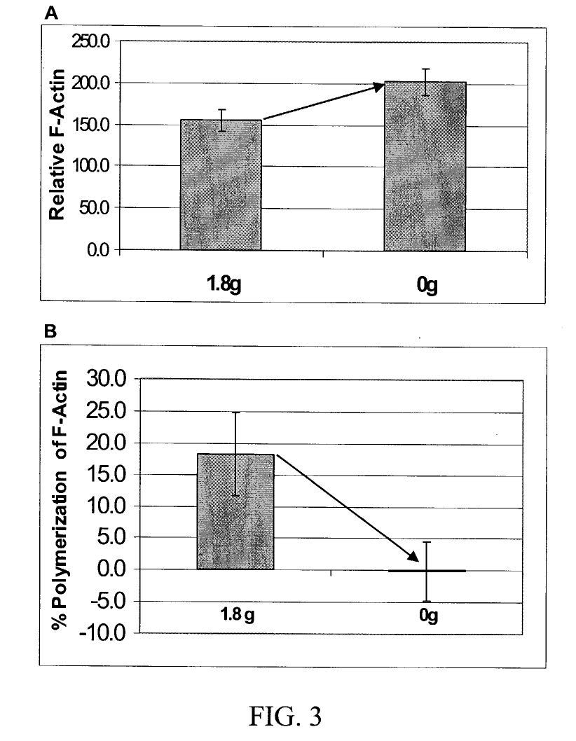

Research over the past few years has shown that cellular behaviors may be dramatically altered under different gravitational loadings. For example, when cells are exposed to lower gravitational loading (e.g., microgravity culture; Hashemi, 1999, FASEB J. 13: 2071-2082) or hyper gravity (e.g., centrifugation), their responses to stimulating agents are altered. FIG. 3A shows that background F-actin contents in Jurkat T cells are higher at microgravity (0 g gravity in a NASA KC-135 parabolic flight) than those at hyper gravity (1.8 g gravity in a parabolic flight on a NASA KC-135 airplane), while FIG. 3B shows that the inducible actin polymerization is dramatically reduced at microgravity (−5±10%) as compared with that at hyper gravity (18±13%). (Hashemi, 1997, Mol. Biol. Cell, 8: 360). Therefore, purification of specific cell types by centrifugation, as performed in prior art methods, can have a significant impact on cellular F-actin contents, which in turn affect cellular responses to stimuli.

To minimize the introduction of artifacts in assessing the responsiveness of blood cells, it is important to minimize manipulation following sample collection from donors. For example in assessing T-cell activation or response of T-cells to stimuli, the use of isolation techniques which expose cells to high g forces by centrifugation should be avoided. In addition, because cells are sensitive to temperature and calcium concentrations, in order to minimize the artifacts associated with these parameters, the culture environment should resemble the in vivo environments as closely as possible by maintaining temperature and calcium concentration throughout the process, and preferably without any purification procedures, e.g., use whole blood, instead of isolated cell subpopulations.

Although there have been studies using whole blood samples to measure F-actin contents in neutrophils (Chen et. al,. 1996, Int'l Arch. Allergy Immunol., 110: 325-331; Tabor et. al., 1998, Kidney Int'l, 53: 783-89; Kimura et al., 1997, J. Immunol. Methods, 202(1): 59-66), these studies focus on a single cell subtype (neutrophils) in the whole blood and use non-physiological conditions to fix the cells. As shown in FIGS. 1 and 2, such conditions introduce artifacts and errors into the F-actin measurements. In addition, these prior art methods do not measure F-actin contents in multiple cells simultaneously.

Methods according to embodiments of the invention may be used to simultaneously measure F-actin contents in multiple cell types in a mixture of cells and these methods are performed in a temperature range close to the normal physiological temperatures, e.g., 30° C.-40° C., preferably around 37° C., to avoid artifacts. Simultaneous measurements as used herein refer to measurements of F-actin contents in several cell types in a mixture of cells without having to purify each cell type, i.e., “simultaneousness” does not mean chronologically at the same time. According to the methods of the invention, all reagents and solutions are preferably pre-equilibrated to a selected temperature, e.g., 37° C., although temperatures in a range from 25° C.-40° C. are expressly within the scope of the present invention. Blood samples are collected in tubes containing one or more non-chelating anticoagulants, e.g., heparins or heparinoids, maintained at or near the selected temperature. Similarly, the assay tubes may be pre-equilibrated at the selected temperature before the assay.

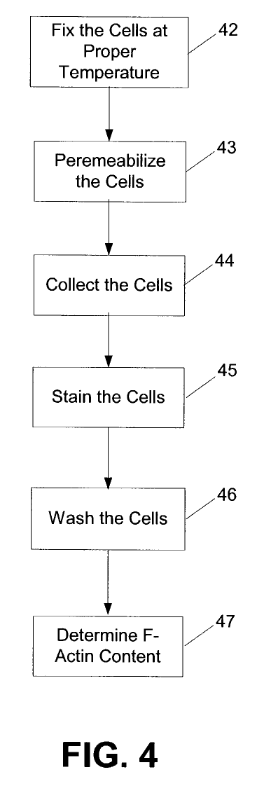

FIG. 4 shows a schematic of a method in accordance with one embodiment of the invention. First, an aliquot of blood sample (e.g., 100 ul) is placed into each of a set of assay tubes having a given dimension (e.g., 12×75 mm). One of ordinary skill in the art would know that the sizes/dimensions of the tubes are not important and should not limit the present invention. Note that both the blood samples and the assay tubes preferably have been pre-equilibrated at the selected temperature, e.g., 37° C. The cells (blood sample) are then fixed by adding a selected amount (e.g., 900 ul) of a fixative solution to each assay tube (step 42). One skilled in the art would appreciate that a fixative solution is buffer solution (e.g., phosphate-buffered saline, PBS) containing one or more cell fixation reagents (e.g., formaldehyde or glutaraldehyde) at a suitable concentration (e.g., 3-5%). The fixative solution may also contain a membrane permeabilization agent, such as saponin or other surfactants/detergents. An exemplary fixative solution may comprise 3.7% formaldehyde and 0.1% saponin in PBS.

The assay tubes are then incubated at the selected temperature, e.g., 37° C., for a selected period of time (e.g., 20 minute) to achieve fixation of the cells. Afterwards, a suitable amount (e.g., 2 ml) of a permeabilization solution may be added to each assay tube at room temperature (25° C.) or at the selected temperature (step 43). In some embodiments, fixation (step 42) and permeabilization (step 43) may be achieved in a single step. A permeabilization solution typically comprises a surfactant (e.g., saponin or other surfactants/detergents—triton, alkyl glucosides, etc.) in a buffer (e.g., PBS or other buffers). The solution may further comprise a preservative or oxidation inhibitor (e.g., sodium azide). An exemplary permeabilization solution may comprise 0.1% saponin and 0.01% sodium azide in PBS.

The samples are then centrifuged at a selected centrifugal force (e.g., 300 g) for a suitable period of time (e.g., 10 minutes) to sediment the cells (step 44). One skilled in the art would appreciate that other combinations of centrifugal force and duration may be used to achieve the same result. The supernatants are removed, for example by decanting, siphoning, or by suction. A suitable amount (e.g., 100 ul) of staining solution, which is prepared from an actin-labeling solution and a cell-phenotyping solution in an appropriate ratio, is then added to each assay tube containing the sedimented cells (step 45).

A cell-phenotyping solution as used herein refers to a solution containing reagents that can bind to specific cell types. A cell type-specific reagent typically comprises a reporter moiety (e.g., a fluorophore, a chromophores, a radio isotope, or an affinity ligand—such as biotin, glutathione, or an oligonucleotide—that can be specifically detected by addition of a labeled reagent—such as avidin/strepavidin, glutathione S-transferase, or a complementary oligonucleotide) to facilitate its detection and an antibody that binds to cell-specific membrane proteins. For example, CD56 molecules are typically found on neural cells, tumors, and lymphocytes that mediate non-MHC-restricted cytotoxicity (MHC stands for major histocompatibility). Thus, a reagent (or antibody) binds specifically or preferentially to CD56 may be used to specifically label this subpopulation of lymphocytes. Similarly, CD3 molecules are typically found on mature T lymphocytes (T cells) and these molecules associate with T-Cell receptors (TCR); hence, antibodies against CD3 may be used to label this population of T cells. CD14 is a glycolipid-anchored membrane glycoproteins expressed on cells of the myelomonocyte lineage, including monocytes, macrophages, and some granulocytes. Thus, antibodies against CD14 are useful for labeling these types of cells. Commonly used reporting moieties on cell-type specific antigens (or reagents) are typically fluorescent molecules so that the labeled cells can be detected using either a fluorometer or a fluorescence-activated cell sorter (FACS). Examples of fluorescent cell-specific labeling reagents include those sold by Beckon Dickinson and Company (Franklin Lakes, N.J.) under the trade names of perCP-CD3™ and APC-CD14™.

Other types of reporter moieties may also be used, e.g., biotin, oligonucleotide, radio-labeling, chromophores, affinity ligands, etc. An affinity ligand reporter moiety may be detected by addition of another reagent, which is in turn labeled with a reporting group, that bind specifically to the affinity ligand. Examples of commonly used affinity ligands may include biotin, glutathione, an oligopeptide, and an oligonucleotide. These affinity ligands can be respectively detected with avidin/strepavidin, glutathione S-transferase (GST), an antibody to the oligopeptide, and a complimentary oligonucleotide. An oligonucleotide affinity ligand may be a synthetic oligonucleotide or a naturally occurring oligonucleotide; they can be DNA (deoxyribonucleic acid), RNA (ribonucleic acid), or the like (e.g., peptide nucleic acid, PNA). An oligonucleotide should have a sufficient length such that the binding to its complementary oligonucleotide will be stable at the temperature used for the experiments; typically, 10-mers or longer.

An actin-labeling solution as used herein refers to a solution containing one or more reagents that can bind specifically or preferentially to actin molecules (G-actin or F-actin, or both), as oppose to other molecules. Reagents that preferentially bind to actin molecules include cytochalasin D, phalloidin, and phallacidin. Cytochalasin D binds to the plus ends of F-actin filaments and prevents further addition of G-actin. Phalloidin and phallacidin are cyclic peptides from the Death Cap fungus (Amanita phalloides); they bind to and stabilize F-actin filaments. These reagents each are typically coupled to a reporter moiety to facilitate their detection. A reagent that contains an F-actin binding moiety and a reporter moiety will be referred to herein as an “F-actin probe.” A reporter moiety of an F-actin probe may include, for example, a fluorophore, a chromophores, a radio isotope, or an affinity ligand—such as biotin or an oligonucleotide—that can be specifically detected by addition of a labeled reagent—such as avidin or the complementary oligonucleotide. Commonly used fluorophore may include NBD, 7-nitrobenz-2-oxa-1,3-diazol-4-yl; FITC, fluorocein isothiocyanate; and BODIPY™, 4,4,-difluoro-3a,4a-diaza-s-indacene. Molecular Probes, Inc. (Eugene, Oreg.) offers various labeled phalloidin and phallacidin, including those under the trade names of BODIPY™-phalloidins and BODIPY™-phallacidins with different excitation and emission wavelengths. An exemplary F-actin labeling (F-actin probe) solution may be prepared by drying 30 ul of a methanol stock solution of BODIPY™-phallacidin, which has been prepared according to the instructions from the supplier, in a glass tube, followed by addition of 1200 ul of the permeabilization solution as described above. An aliquot (e.g., 100 ul) of this actin-labeling solution is then used for each assay tube.

The cells are suspended in the staining solution by tapping (or mixing, shaking, or the like) the tubes, followed by a period of incubation time (e.g., 30 minutes) for labeling of F-actin to occur. A selected amount (e.g., 2 ml) of a wash solution, preferably PBS containing 0.1% saponin and 0.01% sodium azide, is then added to each assay tube followed by centrifugation (e.g., 10 minutes at 300 g) to sediment the cells. Again, the supernatants are discarded, for example by decantation, and the cells re-suspended in a suitable amount (e.g., 300 ul) storage solution (step 46). The samples are then analyzed by flow cytometry for the measurements of intracellular levels of F-actin in cellular subsets (step 47). There are several commercially available flow cytometers (FACS instruments); they are not part of the invention and should not limit the present invention. One exemplary flow cytometer is sold by Becton Dickinson and Company (Franklin Lakes, N.J.) under the trade name of FACSCalibur™.

In addition to flow cytometry, other analytic methods may also be used to obtain the desired information. For example, the labeled cells might be prepared on microscope slides and the labeled cells are examined under a microscope. Similarly, embodiments of the invention may be adapted to miniature assay formats (e.g., 96-wells plates, chips, etc.). Furthermore, various addition and wash steps as described above may be performed automatically by machines.

In addition to flow cytometry or fluorescence microscope, the labeled cells may be measured using a fluorescence spectrometer. In particular, the ratios of fluorescence intensities measured at different wavelengths (at one wavelength for cell type marker and the other wavelength for the F-actin dye) may be used to derive the desired information. Similarly, one skilled in the art would appreciate that fluorescence is but one of the several means that can be used to facilitate the detection. Other alternatives may include chromophores and radio isotopes.

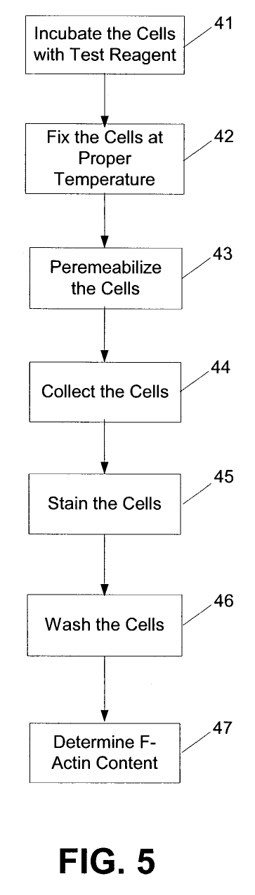

The above example describes one method of F-actin measurements using whole blood in accordance with one embodiment of the invention. Other embodiments of the invention may be used in the study of cellular responses from other cells or of cellular responses to various treatments (e.g., exposure to antigens, chemotatic agents, etc.). FIG. 5 illustrates a schematic of one such example. In this embodiment, an extra step (step 41) is inserted before the step of fixing the cells (step 42), permeabilizing the cells (step 43), collecting the cells (step 44), staining the cells (step 45), washing the cells (step 46), and determining the F-actin contents (step 47) as shown in FIG. 4. This extra step (step 41) involves incubating the cells in a suitable buffer, such as Hanks' buffer—130 mM NaCl, 5.4 mM KCl, 5.5 mM HEPES (4-(2-hydroxyethyl)-1-piperazine ethanesulfonic acid), 5.5 mM glucose, 1.25 mM CaCl₂, and 1.0 mM MgCl₂, with or without a cellular stimulus (e.g., a chemotatic peptide, FMLP, N-formyl-methionyl-leucyl-phenylalanine; protein kinase C activator, phorbol myristate acetate, PMA; anti-TCR/CD3 mAb (monoclonal antibody); lectins; lipopolysacharides, LPS; calcium ionophores, A23187 or the like; bead-immobilized ligands, etc). The reagent's stimulant effects can then be calculated from the difference between the F-actin measurements, obtained from samples incubated with and without the stimulus, according to the methods of the invention. This method can be used to study whole blood samples that are activated with a number of reagents specific for the various cell types.

In addition, other dyes can be used in conjunction with the F-actin measurement techniques disclosed here to correlate multiple parameters from each cell. For example, propidium iodide, which binds preferentially to double-stranded DNA, can be used to correlate cell cycle distribution with F-actin response in each cellular subset. Due to its ability to intercalate into double-stranded DNA, propidium iodide can be used in ploidy analysis, hence cell cycle analysis (A. Krishan, 1975, J. Cell. Biol. 66:188-193). Similarly, propidium iodide (or other agents) may also be used to study cell death (apoptosis) and to correlate apoptosis with cytoskeleton changes as reflected in F-actin measurements. Cell cycles and apoptosis are examples of cellular responses that can be correlated with F-actin measurements according to methods of the present invention. One skilled in the art would appreciate that other cellular responses may also be studies and correlated with the F-actin measurements according to methods disclosed herein.

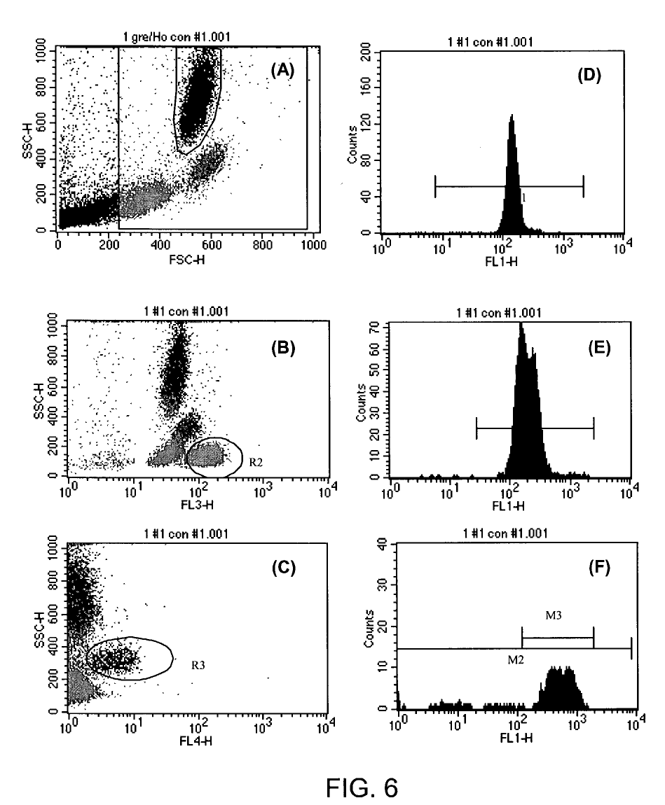

FIGS. 6A-6F show a typical data collection and analysis by flow cytometry. Whole blood samples were cultured and activated at 37° C., followed by fixation at 37° C. Blood samples were then labeled according to the procedures described in FIG. 5 and analyzed by flow cytometry. Data shown here were collected on a Beckon Dickinson FACSCalibur™. This experiment demonstrates the gating techniques used to determine F-actin content of each cellular sub-population. Three gates have been defined to select for different cells: neutrophils are selected based on the Side Scatter/Forward Scatter histogram (FIG. 6A); T-cells are selected based on the CD3 channel (FIG. 6B); and monocytes are selected based on the CD14 channel (FIG. 6C). The circled areas in these figures indicate the selected populations. The actin content of each cell type is then measured using the actin channel and the fluorescence level of each cell is displayed on the corresponding histogram of each cell type (FIGS. 6D-6F, respectively). In this manner, the relative mean fluorescence associated with the F-actin content of each cell population is calculated using statistical analysis of the data. These data are from a single assay tube and the statistics window for each cell type shows the average F-actin content of cells in channel units. This set of data shows the F-actin contents of T-cells, monocytes, and neutrophils. Other cellular sub-populations can readily be analyzed with this technique, provided that an antibody that can specifically bind the fixed cell sub-population for gating purposes is available. For example, probes for CD4 and CD8 may be used to further differentiate the helper and cytotoxic T cells subgroups, respectively.

To improve measurement reliability, each blood sample may be cultured, fixed, labeled and analyzed by flow cytometry in duplicate or triplicate. The average F-actin fluorescence and the standard error of mean may then be calculated for each data point. For example, an experiment with 6 donors over 4 time points during a 24 day period will consist of 4 blood collections per donor. In one example, 90 ul of blood from each blood sample is cultured in each of six assay tubes at 37° C. Three of the assay tubes receive the activator (for example, OKT3 to activate the T-cells) and the other three are used as control. That is, the experiment is performed in triplicate. After sample processing (e.g., fixation, washing, and labeling according to the procedures described in FIG. 5), each tube is analyzed by flow cytometry to generate one data point for each cellular subpopulation.

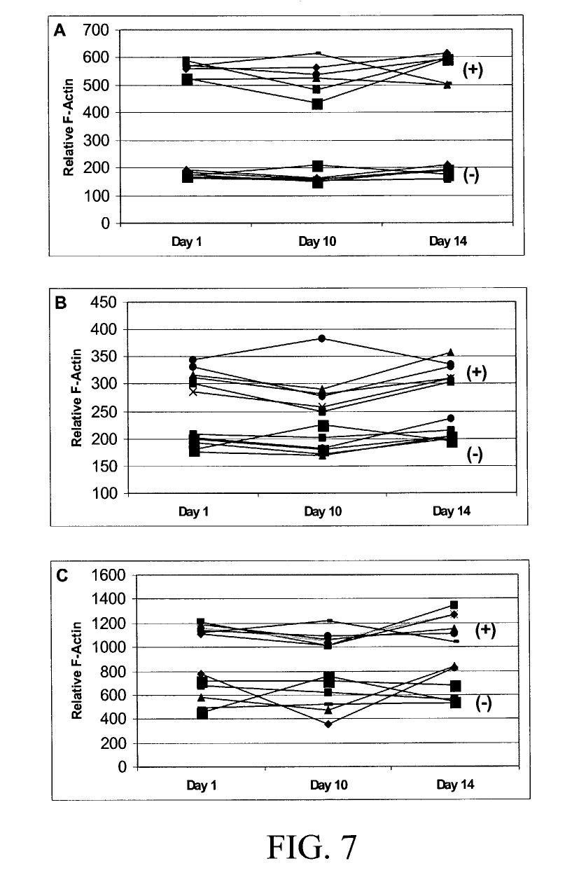

With methods of the invention, convenient measurements of F-actin contents in various cells may be performed with multiple cells without having to purify each cells. These methods make it possible to study F-actin changes/differences in various cells as a function of time or among individuals. For example, FIGS. 7A-7C show F-actin content changes in neutrophils, T cells, and monocytes, respectively, for 6 test subjects over a period of 14 days. As shown in FIGS. 7A-7C, it is possible to follow the F-actin contents and to monitor the inducible F-actin contents of each cell type for each test subject over time. As shown in FIG. 7A, neutrophil F-actin contents are significantly induced by a cellular stimulant (e.g., OKT3) ((+) indicates with the stimulant and (−) indicates without the stimulant). Similarly, FIGS. 7B and 7C show that F-actin contents in T cells and monocytes, respectively, are also inducible ((+) indicates with the stimulant and (−) indicates without the stimulant), albeit to a lesser extent.

With the ability to determine cell F-actin contents as “molecular signatures” of various cell types, it becomes possible to monitor cell exposures to chemical, biological, or environmental factors. Thus, embodiments of the invention may be useful in screening possible terrorist attacks.

While the above described methods involve fixing the cells, embodiments of the invention may also be used without fixing the cells. For example, if it is desirable to monitor live cell F-actin contents or to monitor live cell responses to stimuli, cells should not be fixed.

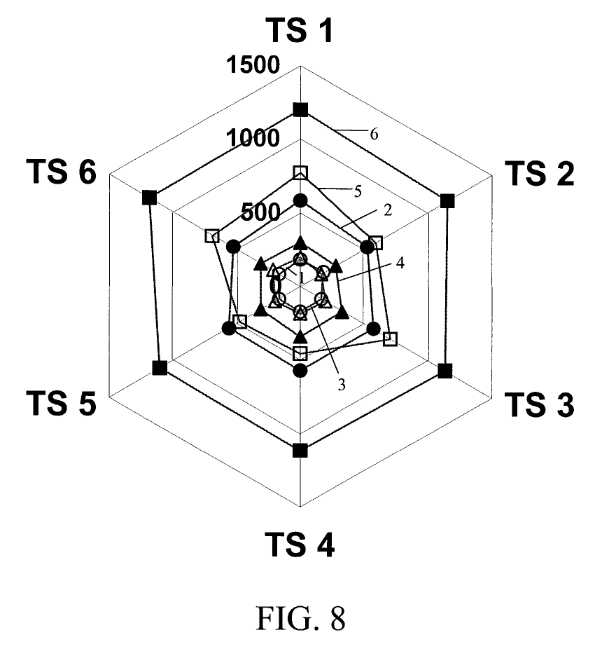

In addition to monitoring F-actin signatures of a particular subject as a function of time, methods of the invention are also useful in comparing F-actin signatures of a large number of subjects. FIG. 8 shows that F-actin contents in various cells from the various donors (test subjects, TS) can be readily compared, for example, by plotting on the same graph for each time point to determinate donor-to-donor variability. Curves 1-6 represents F-actin contents of neutrophils without a stimulant, neutrophils with a stimulant, T cells without a stimulant, T cells with a stimulant, monocytes without a stimulant, and monocytes with a stimulant, respectively. As shown in FIG. 8, while the neutrophils (curves 1 and 2, without and with stimulation, respectively) of this test subject population show similar levels of F-actin in the cells, the monocytes (curves 5 and 6, without and with stimulation, respectively) of TS 1, 5, and 6 have much higher F-actin contents.

This data set in FIG. 8 demonstrates the application of the methods of the invention to a small group of donors. If a large number of samples are available, it would be possible to establish “normal” distributions for each cellular subset as a baseline for analysis of data from a specific study—the baseline F-actin signature. In other words, the “normal” distributions of F-actin contents in each cellular subset may be used in other studies, for example, in patient diagnosis. In addition, the “normal” F-actin contents in various cells may be used as “molecular signatures” for screening general populations for disease outbreaks, and effects of chemical or biological warfare/terrorism.

One advantage of the present invention includes the ability to determine intracellular F-actin contents with minimal disturbance because the cells are fixed and permeabilized at a temperature at or near the physiological temperature. This process allows preservation of cellular architecture without artifacts due to temperature shifts. In addition, cells are not subjected to centrifugal forces before they are fixed; this eliminates the unnecessary artifacts due to gravity changes. Methods of the invention also avoid alteration of calcium concentrations by collecting blood samples into non-chelating coagulants.

Advantageously, embodiments of the present invention, which allow simultaneous measurements of F-actin in blood cellular subsets (e.g., T-cells, monocytes, NK cells, Neutrophils, etc.), provide unique tools for rapidly assessing the status of blood cells and their responsiveness using the F-actin levels as the molecular signature of each cellular subset. These methods can be applied in clinical research and patient diagnostics where an evaluation of the status of blood cells and their responsiveness to stimulating agents are useful for diagnosis of disease and evaluation of treatment protocols. It is also valuable in situations where evaluation of responsiveness of blood cells is needed. Applications of this technology further include: studies of cellular responses to stimulating agents (e.g., anti-TCR/CD3 mAb, lectins, Lipopolysacharides (LPS), phorbol esters, calcium ionophores, bead-immobilized ligands, FMLP, etc), such as immune enhancement or suppression by immuno-responsive agents (e.g., cytokines), chemotaxis in responses to chemotatic agents (e.g., FMLP), therapeutic responses (e.g., AIDS treatments, cancer chemotherapy), diagnostics tool for drug discovery application, assessment of disease in patient populations (such as Wiskott-Aldrich Syndrome, cancer, AIDS virus infection, and immune disorders such as autoimmune rheumatoid arthritis), and the response of patients to treatment, evaluation of toxicity due to exposure to chemical or biological agents (e.g., screening of large populations for bioterrorism), assessment of transplant reactivity utilizing blood samples from recipients and tissue samples from donors to assess cellular reactivity, and the like.

While the invention has been described with respect to a limited number of examples, those skilled in the art, having benefit of this disclosure, would appreciate that other embodiments can be devised which do not depart from the scope of the invention as disclosed herein. Accordingly, the scope of the invention should be limited only by the attached claims

What is claimed is:

1. A method for F-actin measurement in a mixture of cells, comprising:
fixing the mixture of cells at a selected temperature;
labeling at least one type of cell in the mixture of cells using cell type-specific reagents;
labeling F-actin using an F-actin probe; and
determining a content of the F-actin in each of the at least one type of cell in the mixture of cells.

2. The method of claim 1, wherein the mixture of cells comprises whole blood.

3. The method of claim 2, wherein the at least one type of cell comprises one selected from the group consisting of T lymphocytes, neutrophils, and monocytes.

4. The method of claim 2, wherein the whole blood is collected using a non-chelating anticoagulant.

5. The method of claim 4, wherein the non-chelating anticoagulant comprises heparin.

6. The method of claim 1, wherein the cell type-specific reagent comprises a reporter moiety and an antibody.

7. The method of claim 6, wherein the reporter moiety comprises one selected from the group consisting of a fluorophore, a chromophores, a radio isotope, and an affinity ligand.

8. The method of claim 7, wherein the affinity ligand comprises one selected from the group consisting of biotin, glutathione, an oligopeptide, and an oligonucleotide.

9. The method of claim 1, wherein the F-actin probe comprises a reporter moiety and an actin-specific binding reagent.

10. The method of claim 9, wherein the actin-specific binding reagent comprises one selected from the group consisting of cytochalasin D, phalloidin, and phallacidin.

11. The method of claim 9, wherein the reporter moiety comprises one selected from the group consisting of a fluorophore, a chromophores, a radio isotope, and an affinity ligand.

12. The method of claim 11, wherein the affinity ligand comprises one selected from the group consisting of biotin, glutathione, an oligopeptide, and an oligonucleotide.

13. The method of claim 1, wherein the determining the content of the F-actin is performed using a fluorescence activated cell sorter.

14. The method of claim 1, wherein the fixing the mixture of cells is performed with a solution comprising saponin.

15. The method of claim 1, wherein the selected temperature is in a range of 30 to 40 degrees Celsius

16. The method of claim 1, wherein the selected temperature is about 37 degrees Celsius.

17. A method for measuring an effect of a cellular stimulant in a mixture of cells, comprising:
incubating an experimental sample of the mixture of cells with the cellular stimulant in a selected buffer at a selected temperature;
incubating a control sample of the mixture of cells in the selected buffer without the cellular stimulant at the selected temperature;
fixing the experimental sample and the control sample separately at the selected temperature;
labeling at least one type of cell in the experimental sample and the control sample separately using cell type-specific reagents;
labeling F-actin in the experimental sample and the control sample separately using an F-actin probe;
determining a content of the F-actin in each of the at least one type of cell in the experimental sample and the control sample separately; and
comparing the content of the F-actin in the each of the at least one type of cells in the experimental sample with the content of the F-actin in a corresponding cell in the control sample.

18. The method of claim 17, wherein the mixture of cells comprises whole blood.

19. The method of claim 18, wherein the at least one type of cells comprises one selected from the group consisting of T lymphocytes, neutrophils, and monocytes.

20. The method of claim 18, wherein the whole blood is collected using a non-chelating anticoagulant.

21. The method of claim 20, wherein the non-chelating anticoagulant comprises heparin.

22. The method of claim 17, wherein the cell type-specific reagent comprises a reporter moiety and an antibody.

23. The method of claim 22, wherein the reporter moiety comprises one selected from the group consisting of a fluorophore, a chromophores, a radio isotope, and an affinity ligand.

24. The method of claim 23, wherein the affinity ligand comprises one selected from the group consisting of biotin, glutathione, an oligopeptide, and an oligonucleotide.

25. The method of claim 17, wherein the F-actin probe comprises a reporter moiety and an actin-specific binding reagent.

26. The method of claim 25, wherein the actin-specific binding reagent comprises one selected from the group consisting of cytochalasin D, phalloidin, and phallacidin.

27. The method of claim 25, wherein the reporter moiety comprises one selected from the group consisting of a fluorophore, a chromophores, a radio isotope, and an affinity ligand.

28. The method of claim 27, wherein the affinity ligand comprises one selected from the group consisting of biotin, glutathione, an oligopeptide, and an oligonucleotide.

29. The method of claim 17, wherein the determining the content of the F-actin is performed using a fluorescence activated cell sorter.

30. The method of claim 17, wherein the fixing is performed with a solution comprising saponin.

31. The method of claim 17, wherein the selected temperature is in a range of 30 to 40 degrees Celsius

32. The method of claim 17, wherein the selected temperature is about 37 degrees Celsius.
